(19) United States
(12) Patent Application Publication
(10) Pub. No.: US 2026/0365687 A1
(43) Pub. Date: Nov. 19, 2026

(54) ORGANIC COMPOUNDS FOR PREVENTING AND/OR TREATING OCULAR DISORDERS

(71) Applicant: Aihol Corporation, DIAMOND BAR, CA (US)

(72) Inventor: Tzu-Yang CHEN, Taipei City (TW)

(73) Assignee: Aihol Corporation, DIAMOND BAR, CA (US)

(21) Appl. No.: 19/652,836

(22) Filed: Apr. 20, 2026

Related U.S. Application Data
(60) Provisional application No. 63/792,270, filed on Apr. 21, 2025.

(51) Int. Cl. C07H 3/06; A61K 31/702

(52) U.S. Cl. CPC C07H 3/06; A61K 31/702

(57) ABSTRACT

Disclosed herein are compositions and methods for the treatment of ocular disorders. According to some embodiments, the compositions and methods involve the use of an organic compound.

### 1. FIELD OF THE INVENTION

The present disclosure relates to compositions and methods for preventing and/or treating ocular disorders in a patient. More particularly, the present compositions and methods involve the use of organic compounds of formula 1 in the treatment and/or prevention of ocular disorders.

### 2. DESCRIPTION OF RELATED ART

Ocular disorders include inflammatory ocular disorders and angiogenic ocular disorders. Dry eye syndrome, diabetic retinopathy (DR), age-related macular degeneration (AMD), and uveitis are typical examples of inflammation-related ocular disorders. Angiogenic ocular disorders involves neovascularization within the eye, which contributes to moderate to severe vision loss in several ocular diseases, including diabetic retinopathy, retinal vein occlusions, retinopathy of prematurity, wet age-related macular degeneration (wet AMD, also known as neovascular AMD (nAMD) or exudative AMD), ocular histoplasmosis, pathologic myopia, and other related diseases.

AMD is the leading cause of irreversible severe vision loss among people age 50 and older. AMD includes dry age-related macular degeneration (dry AMD) and wet AMD. Dry AMD accounts for approximately 80%-90% of cases and causes blurred or reduced central vision due to the breaking down of the inner layers of the macula. wet AMD is a serious type of late AMD, which happens when vascular endothelial growth factor (VEGF) induces abnormal blood vessels growth in the retina. The current standard treatment for wet AMD involves an injection of anti-VEGF agent into the vitreous chamber inside the eye. Anti-VEGF agents can slow or stop the growth of the leaky blood vessels and, in most cases, effectively stave off further vision loss. Common anti-VEGF agents used to treat wet AMD include aflibercept (EYLEA®), brolucizumab (BEOVU®), faricimab-svoa (VABYSMO®), and ranibizumab (LUCENTIS®).

However, anti-VEGF treatments are very expensive. For example, the cost for Eylea for the first year is around 18,000 U.S. dollars per eye. Therefore, there exists a need in the related art to provide additional, effective compositions and methods for preventing and/or treating ocular disorders.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present disclosure is directed to a method for preventing and/or treating ocular disorders, in a patient in need thereof.

According to some embodiments of the present disclosure, the method comprises the step of administering to the patient an effective amount of an organic compound.

In certain embodiments, an organic compound represented by the following formula 1:

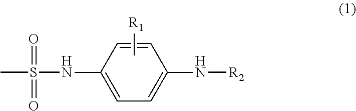

where R₁ represents a phenyl group, a phenoxy group, a (C1-C30) alkyl group, a (C2-C30) alkenyl group, a (C2-C30) alkynyl group or a (C1-C30) alkoxy group; R₂ represents a hydroxyl group, carboxyl group or a saccharide.

In certain embodiments, the saccharide of R₂ comprises one to four saccharide units. For example, the saccharide units may be at least one selected from the group consisting of glucose, fructose, galactose, glucuronic acid, sucrose, lactose, maltose, glycosaminoglycan, isomaltotriose, panose, nigerotriose, maltotriose, melezitose, maltotriulose, raffinose, kestose, ychnose, maltotetraose, nigerotetraose, nystose, sesamose and stachyose, and derivatives thereof. In certain embodiment, the saccharide unit is a glycosaminoglycan composed of at least one disaccharide unit of a D-glucuronic acid and an N-acetyl-D-glucosamine (-4GlcUAβ1-3GlcNAcβ1-).

In certain embodiments, the organic compound of formula 1 is administered to the patient via an intravitreal injection (IVT).

The organic compounds according to the present disclosure include, but are not limited to, the following compounds:

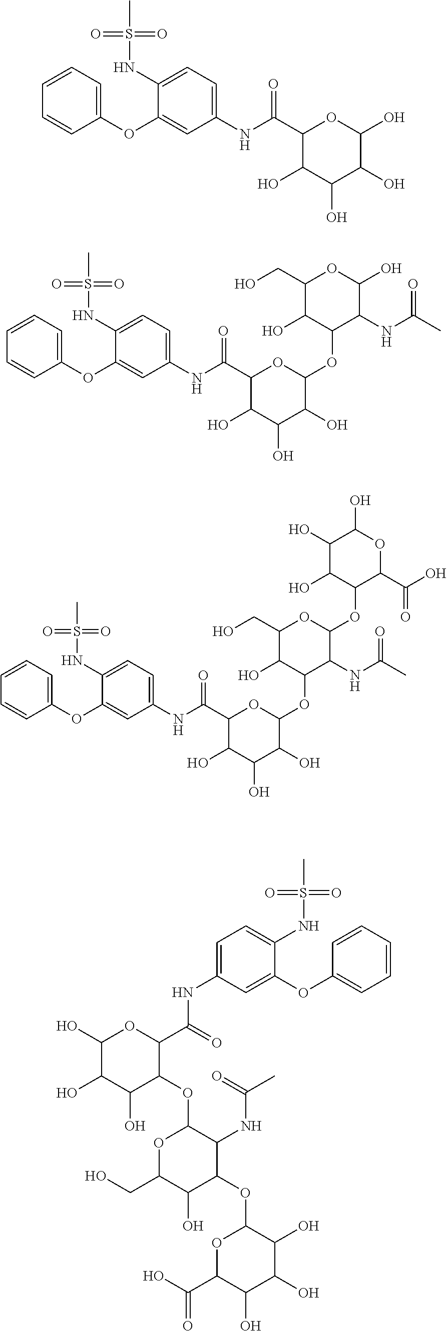 [SMILES: CS(=O)(=O)Nc1ccc(NC(=O)C2OC(O)C(O)C(O)C2O)cc1Oc1ccccc1 ; CC(=O)NC1C(O)OC(CO)C(O)C1OC1OC(C(=O)Nc2ccc(NS(C)(=O)=O)c(Oc3ccccc3)c2)C(O)C(O)C1O ; CC(=O)NC1C(OC2C(C(=O)O)OC(O)C(O)C2O)OC(CO)C(O)C1OC1OC(C(=O)Nc2ccc(NS(C)(=O)=O)c(Oc3ccccc3)c2)C(O)C(O)C1O ; CC(=O)NC1C(OC2C(C(=O)Nc3ccc(NS(C)(=O)=O)c(Oc4ccccc4)c3)OC(O)C(O)C2O)OC(CO)C(O)C1OC1OC(C(=O)O)C(O)C(O)C1O]

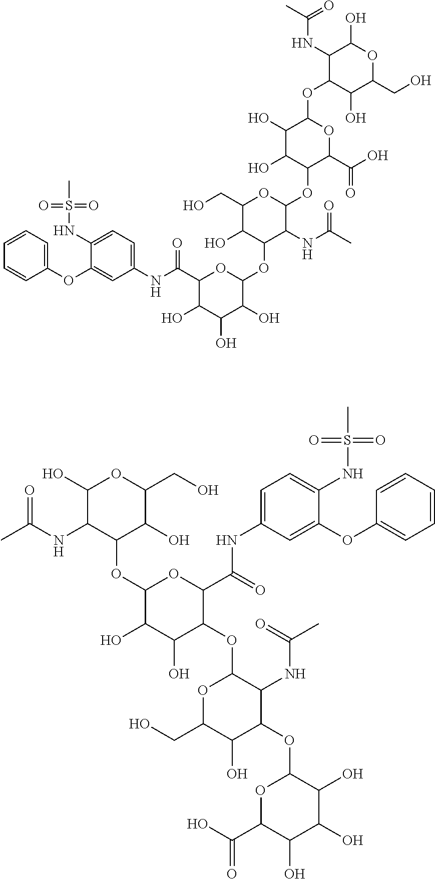 [SMILES: CC(=O)NC1C(O)OC(CO)C(O)C1OC1OC(C(=O)O)C(OC2OC(CO)C(O)C(OC3OC(C(=O)Nc4ccc(NS(C)(=O)=O)c(Oc5ccccc5)c4)C(O)C(O)C3O)C2NC(C)=O)C(O)C1O ; CC(=O)NC1C(O)OC(CO)C(O)C1OC1OC(C(=O)Nc2ccc(NS(C)(=O)=O)c(Oc3ccccc3)c2)C(OC2OC(CO)C(O)C(OC3OC(C(=O)O)C(O)C(O)C3O)C2NC(C)=O)C(O)C1O]

According to some embodiments of the present disclosure, ocular disorders is dry eye syndrome, dry AMD, wet AMD, macular edema, macular edema following retinal vein occlusion, retinal vein occlusion (RVO), central retinal vein occlusion (CRVO), branch retinal vein occlusion (BRVO), diabetic macular edema (DME), choroidal neovascularization (CNV), iris neovascularization, neovascular glaucoma, proliferative vitreoretinopathy (PVR), optic disc neovascularization, corneal neovascularization, or proliferative diabetic retinopathy (PDR).

In another aspect, the present disclosure is directed to a pharmaceutical composition for preventing and/or treating ocular disorders.

According to some embodiments, the pharmaceutical composition comprises an effective amount of an organic compound of formula 1 and a pharmaceutically-acceptable excipient.

Subject matters that are also included in other aspects of the present disclosure include the use of an organic compound of formula 1 in the manufacture of a medicament for use in the prevention and/or treatment of ocular disorders, as well as an organic compound of formula 1 for use in the prevention and/or treatment of ocular disorders.

Many of the attendant features and advantages of the present disclosure will becomes better understood with reference to the following detailed description considered in connection with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present description will be better understood from the following detailed description read in light of the accompanying drawings, where:

FIG. 1 shows representative images of fundus fluorescence angiography after laser induction and intravitreal injection of PBS, Eylea, and HA4-N-(4-Amino-2-phenoxyphenyl)methanesulfonamide (HA4-MSCA), respectively. Five or six laser burns were localized around the optic disc in each eye.

FIG. 2 shows the CNV lesion area ratio (Day 14/Day 7) of the individual lesion in PBS, Eylea, and HA4-MSCA groups in FFA images. All data were analyzed using Tukey's test and reported as the mean±2 times the standard deviation and quartile. The *p<0.05 indicated a significant difference.

FIG. 3 shows the intraocular pressure changed after intravitreal injection of PBS, Eylea, and HA4-MSCA for 7 and 14 days. Data were reported as the mean±standard deviation.

FIG. 4 shows images of the tube formation in HUVECs when treated with two HA4-MSCA according to the present disclosure and a comparative compound, HA10k-MSCA.

FIGS. 5A to 5D are histograms illustrating the total branching length and total segment length of the tubes formed in the presence of HA4-MSCA according to the present disclosure and a comparative compound, HA10k-MSCA.

FIG. 6 shows images of the tube formation in HUVECs when treated with HA2-MSCA (AB8141) at various concentrations according to the present disclosure.

FIGS. 7A and 7B are histograms illustrating the total branching length and total segment length of the tubes formed in the presence of HA2-MSCA (AB8141) at various concentrations according to the present disclosure.

FIG. 8 shows images of the tube formation in HUVECs when treated with HA1-MSCA (AB8198) at various concentrations according to the present disclosure.

FIGS. 9A and 9B are histograms illustrating the total branching length and total segment length of the tubes formed in the presence of HA1-MSCA (AB8198) at various concentrations according to the present disclosure.

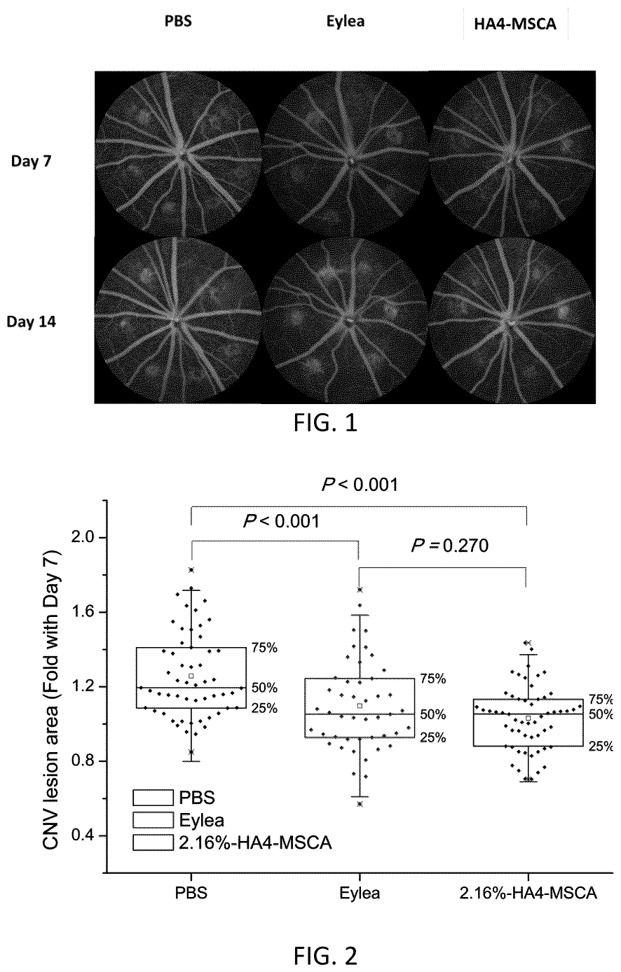

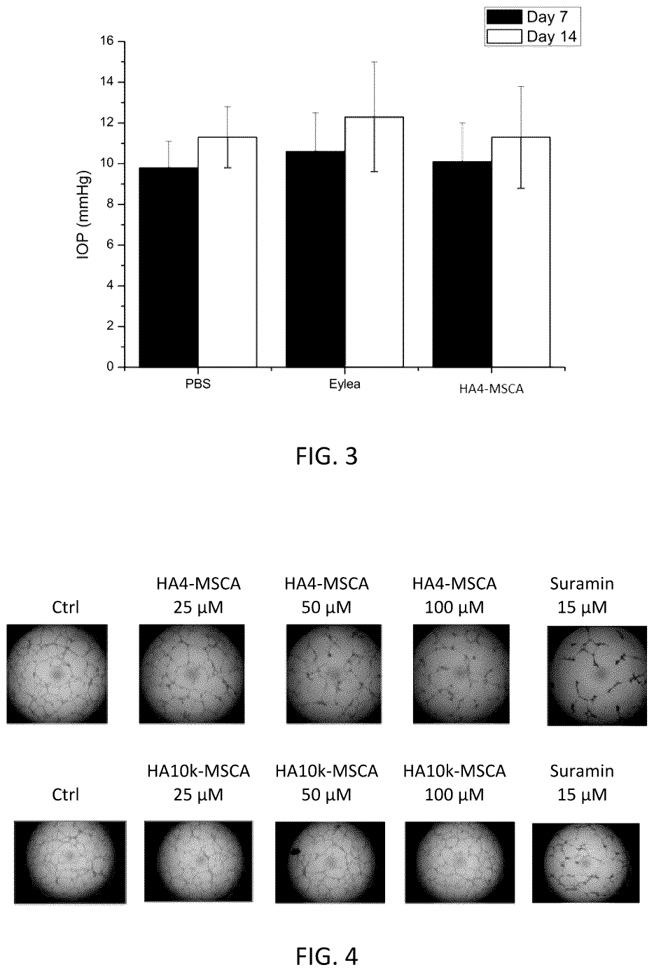

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques. Ranges can be expressed herein as from one endpoint to another endpoint or between two endpoints. All ranges disclosed herein are inclusive of the endpoints, unless specified otherwise.

The terms “treatment” and “treating” as used herein may refer to a preventative (e.g., prophylactic), curative or palliative measure. In particular, the term “treating” as used herein refers to the application or administration of the present organic compound or a pharmaceutical composition comprising the same to a subject, who has a medical condition, a symptom associated with the medical condition, a disease or disorder secondary to the medical condition, or a predisposition toward the medical condition, with the purpose to partially or completely alleviate, ameliorate, relieve, delay onset of, inhibit progression of, reduce severity of, and/or reduce incidence of one or more symptoms or features of said particular disease, disorder, and/or condition. Treatment may be administered to a subject who does not exhibit signs of a disease, disorder, and/or condition, and/or to a subject who exhibits only early signs of a disease, disorder and/or condition, for the purpose of decreasing the risk of developing pathology associated with the disease, disorder and/or condition.

The terms “subject” and “patient” are used interchangeably herein and are intended to mean an animal including the human species that is treatable by the organic compound, pharmaceutical composition, and/or method of the present invention. The term “subject” or “patient” intended to refer to both the male and female gender unless one gender is specifically indicated.

The terms “application” and “administration” are used interchangeably herein to mean the application of an organic compound or a pharmaceutical composition of the present invention to a subject in need of a treatment thereof.

The term “effective amount” as used herein refers to the quantity of the present organic compound that is sufficient to yield a desired therapeutic response. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as particular condition being treated, the physical condition of the patient (e.g., the patient's body mass, age, or gender), the type of mammal or animal being treated, the duration of the treatment, the nature of concurrent therapy (if any), and the specific formulations employed and the structure of the therapeutic agent or its derivatives. Effective amount may be expressed, for example, as the total mass of the organic compound (or the equivalent amount of the drug), e.g., in grams, milligrams or micrograms, or an effective concentration of the organic compound in the final formulation, e.g., as milligrams per milliliter (mg/mL).

The phrase “pharmaceutically acceptable excipient” as used herein means a pharmaceutically acceptable material, composition or vehicle, such as a liquid, diluent, carrier, solvent, involved in carrying or transporting the subject agents from one organ or portion of the body, to another organ or portion of the body. Each excipient must be “acceptable” in the sense of being compatible with the other ingredients of the formulation. The pharmaceutical formulation contains the organic compound of the invention in combination with one or more pharmaceutically acceptable ingredients. Preferably, the excipient can be in the form of a liquid diluent. These pharmaceutical preparations are a further object of the invention. Usually, the amount of active substance is between 0.1-95% by weight of the preparation, preferably between 0.2-50% by weight in preparations for parenteral use or for topical administration. For the clinical use of the methods of the present invention, the pharmaceutical composition of the invention is formulated into formulations suitable for the intended route of administration.

As used herein, the term “hyaluronic acid” (HA) (also called hyaluronate or hyaluronan) is an anionic, nonsulfated glycosaminoglycan composed of at least one disaccharide unit, specifically a D-glucuronic acid and an N-acetyl-D-glucosamine (-4GlcUAβ1-3GlcNAcβ1-). The molecular of HA can range from 379 Daltons (Da) (basic disaccharide unit of HA) to millions of Daltons. The term “HA derivative” refers to an HA having any modification on the hydroxyl, carboxyl, amide, or acetylamino groups of one or more disaccharide units of the HA.

The present disclosure is based, at least in part, on the discovery that the present organic compounds unexpectedly decrease choroidal neovascularization in rat. In view of the foregoing, the present disclosure proposes methods for preventing and/or treating ocular disorders. Some embodiments of the present disclosure are directed to methods for preventing and/or treating ocular disorders. Also provided herein is the use of said organic compound in the prevention and/or treatment of said ocular disorders, as well as its use in the manufacture of a medicament for said treatment purpose. The medicament (i.e., a pharmaceutical composition) is, of course, a subject matter covered by the scope of the present application.

In one aspect, the present disclosure is directed to a method for preventing and/or treating ocular disorders, in a patient in need thereof.

According to some embodiments of the present disclosure, the method comprises the step of administering to the patient an effective amount of an organic compound of formula 1:

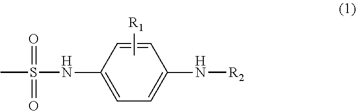

where R₁ represents a phenyl group, a phenoxy group, a (C1-C30) alkyl group, a (C2-C30) alkenyl group, a (C2-C30) alkynyl group or a (C1-C30) alkoxy group; R₂ represents a hydroxyl group, carboxyl group or a saccharide.

In certain embodiments, the saccharide of R₂ comprises one to four saccharide units. For example, the saccharide units may be at least one selected from the group consisting of glucose, fructose, galactose, glucuronic acid, sucrose, lactose, maltose, glycosaminoglycan, isomaltotriose, panose, nigerotriose, maltotriose, melezitose, maltotriulose, raffinose, kestose, ychnose, maltotetraose, nigerotetraose, nystose, sesamose and stachyose, and derivatives thereof. In certain embodiment, the saccharide unit is a glycosaminoglycan composed of at least one disaccharide unit of a D-glucuronic acid and an N-acetyl-D-glucosamine (-4GlcUAβ1-3GlcNAcβ1-).

In certain embodiments, the organic compound of formula 1 is administered to the patient via an intravitreal injection (IVT).

For example, the organic compound can be formulated, together with a pharmaceutically-acceptable excipient, into a pharmaceutical composition suitable for the desired mode of administration. Certain pharmaceutical compositions prepared in accordance with the presently disclosed and claimed inventive concept(s) are single unit dosage forms intravitreal administration to a patient. As could be appreciated, these pharmaceutical compositions are also within the scope of the present disclosure.

Optionally, the pharmaceutical compositions may comprise one or more of the following additives: buffer, thermal stabilizer, viscosity reducing agent, and surfactant, such that the pharmaceutical compositions have a pH and viscosity suitable for injection, and in particular, for therapeutic intravitreal injection.

In certain embodiments, the effective concentration of the organic compound is 1 to 100 mg/mL of the organic compound. Specifically, the effective concentration of the organic compound may be 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/mL of the organic compound.

According to some embodiments of the present disclosure, ocular disorders include dry eye syndrome, dry AMD, wet AMD, macular edema, macular edema following retinal vein occlusion, retinal vein occlusion (RVO), central retinal vein occlusion (CRVO), branch retinal vein occlusion (BRVO), diabetic macular edema (DME), choroidal neovascularization (CNV), iris neovascularization, neovascular glaucoma, proliferative vitreoretinopathy (PVR), optic disc neovascularization, corneal neovascularization, or proliferative diabetic retinopathy. Additional ocular disorders are also subject to treatment using a composition or method disclosed herein.

In another aspect, the present disclosure is directed to a pharmaceutical composition, for preventing and/or treating ocular disorders.

According to some embodiments, the pharmaceutical composition comprises an effective amount of an organic compound and a pharmaceutically-acceptable excipient.

Subject matters that are also included in other aspects of the present disclosure include the use of an organic compound in the manufacture of a medicament for use in the treatment of ocular disorders, as well as an organic compound for use in the treatment of ocular disorders.

Yet another aspect of the present disclosure is direct to the use of an organic compound in the manufacture of a medicament, for use in the treatment of ocular disorders. Still another aspect of the present disclosure is direct to the use of an organic compound, in the treatment of ocular disorders. Similarly, the various dosing regimens (including dosages and dosing intervals) concerning the organic compound, and the various ocular disorders described above are also applicable in these aspects.

The following discussion is provided to elucidate certain aspects of the present invention and to aid those of skilled in the art in practicing this invention. This discussion is in no way to be considered to limit the scope of the invention in any manner. Without further elaboration, it is believed that one skilled in the art can, based on the description herein, utilize the present invention to its fullest extent.

### Working Example 1

### Evaluation of Therapeutic Effect of HA4-MSCA in Laser-Induced CNV Model

In this example, laser-induced CNV model is used to investigate the therapeutic effect of HA tetrasaccharide-N-(4-Amino-2-phenoxyphenyl)methanesulfonamide (HA4-MSCA) in treating CNV.

Two disaccharide units of D-glucuronic acid and N-acetylglucosamine (HA tetrasaccharide; HA4) was reacted with (4-Amino-2-phenoxyphenyl)methanesulfonamide (MSCA) to give HA4-MSCA. Briefly, 50 mg of HA tetrasaccharide (HA4) (0.06437 mmol) was dissolved in 3 mL D.D. Water. 17.7 mg of Oxyma (0.1244 mmol) was dissolved in 1 mL DMSO and then added to the HA4 solution under stirring for 10 minutes; then, the 38.1 mg (0.1368 mmol) (4-Amino-2-phenoxyphenyl)methanesulfonamide (MSCA) was de-protected and then dissolved in 1 mL DMSO and added to the HA4 solution. After stirring for 10 minutes, 29.2 μL of DIC (0.1866 mmol) was dissolved in 4 mL of DMSO under stirring for 1 minute and then added to the HA4 solution. The final reaction mixture was stirred for 24 hours at room temperature, and the resulting solution was freeze dried for three days and purified by reverse phase C18 column for Medium Pressure Liquid Chromatography (MPLC) to give HA4-MSCA.

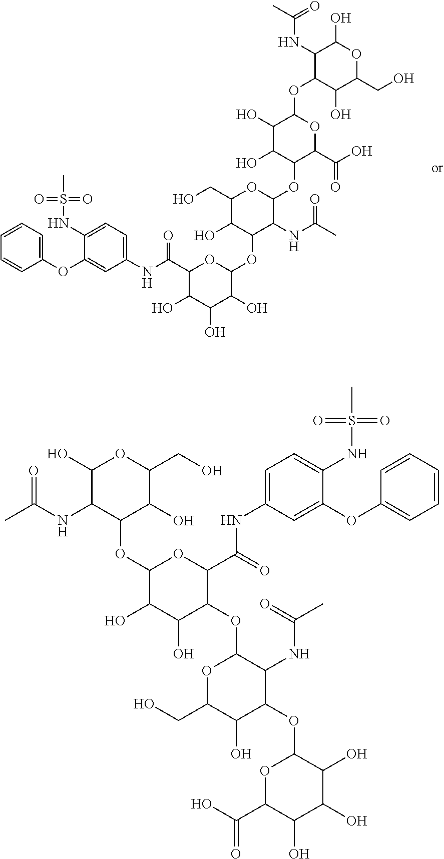 [SMILES: CC(=O)NC1C(O)OC(CO)C(O)C1OC1OC(C(=O)O)C(OC2OC(CO)C(O)C(OC3OC(C(=O)Nc4ccc(NS(C)(=O)=O)c(Oc5ccccc5)c4)C(O)C(O)C3O)C2NC(C)=O)C(O)C1O ; CC(=O)NC1C(O)OC(CO)C(O)C1OC1OC(C(=O)Nc2ccc(NS(C)(=O)=O)c(Oc3ccccc3)c2)C(OC2OC(CO)C(O)C(OC3OC(C(=O)O)C(O)C(O)C3O)C2NC(C)=O)C(O)C1O]

EYLEA® (aflibercept) was provided by Taichung Veterans General Hospital. Fluorescein sodium salt was purchased from Sigma-Aldrich (St Louis, MO, USA). Zoletil 50 was purchased from Virbac (Carros, France). Sutoin was purchased from Juily Pharmaceutical Co. Ltd (Taipei, Taiwan). Alcaine 0.5% was purchased from Alcon (Fort Worth, Texas, USA). Vidisic Gel was purchased from Dr. Gerhard Mann Chem-pharm (Berlin, Germany). Mydrin®-M was purchased from Santen (Niittyhaankatu, Finland).

Brown Norway rats (purchased from Laboratory Animal Center, Taiwan) weighing between 180 and 200 g were used for the study. All rats were housed at a room temperature of 23 °C±2 °C with a relative humidity of 60%±10% and alternating 12-hr light-dark cycles (7 A.M. to 7 P.M.), and all rats were given food and water ad libitum. All procedures were approved by the Institutional Animal Care and Use Committee (IACUC) of Chung Hsing University (IACUC approval no. 109-076^{R} and 111-091).

The laser induction model was performed as follows. After anesthesia and pupil dilatation, six laser burns were induced using a green laser (532 nm, 150 mW, 100 ms) (Oculight® GL Laser, IRIDEX, Mountain View, USA), focused on the retinal pigment epithelium. The presence of a bubble confirms the success of the laser impact. Animals were randomly divided, and the treatment was applied 7 days-post laser induction via an intravitreal injection as follows: (I). 5 μL of PBS, (II). 5 μL of 2.16% HA4-MSCA (equivalent to 28.94 μg of MSCA), (III). 2.2 μL of 0.4% Eylea (8.8 μg of aflibercept). Color photographs, fundus fluorescein angiography (FFA), and flat mount staining were used to detect the lesions in the laser-induced CNV model. ImageJ software was used to select and quantify the area (pixel) of the lesion in FFA.

All solutions were preservative-free and sterilized via the 0.22 μm polyether sulfone filter (PBS and HA4-MSCA). All the procedures were performed under general anesthesia and administered via an intraperitoneal injection of Sutoin and Zoletil 50 (2:1 ratio, 1 mL/kg of the mixed solution) and topical administration of 0.5% Alcaine and Mydrin©-M.

For the flat mount, the whole eyeball was fixed in the 4% formaldehyde at room temperature for an hour. The anterior segment was removed from the enucleated eyeball, and retinal and retinal pigment epithelium-choroid-sclera (RCS) complex was carefully separated from the remaining eye cup sample. After washing with PBS solution, the RCS complex was incubated in blocking solution (0.1% Triton x-100, 4% BSA in PBS), and Isolectin B4 was applied to RCS complexes overnight at 4 °C in blocking solution. After washing with PBS five times, the RCS complexes were examined using a fluorescence microscope (FV1200, Olympus, Tokyo, Japan).

The quantitative data are presented as mean±2×standard deviation and quartile. Data were analyzed using Tukey's one-way tests to perform comparisons, and a p-value <0.05 was considered to indicate a statistically significant difference.

To confirm the therapeutic effect on CNV formation, fundus imaging and fluorescein fundus angiography were performed on days 7 and 14 after laser treatment. As shown in, the CNV lesions formed around the optic disc in all groups after 7 days of laser induction. The quantification data were analyzed and shown as CNV lesion area ratio via individual calculation in. Both the Eylea and HA4-MSCA treatment significantly reduced the neovascularization progress compared to the PBS administration. However, no significant difference was observed between the Eylea and HA4-MSCA administration.

For safety assessment, the intraocular pressure during the observation period and after intravitreal injection was measured. As shown in, no significant changes were observed in comparison to the PBS group for Eylea and HA4-MSCA.

### Working Example 2

### Evaluation of Therapeutic Effect of HA4-MSCA and HA10k-MSCA on Human Umbilical Vascular Endothelial Cell Tube Formation

HUVEC human umbilical vein endothelial cell line (C2519A), EGMTM-2 (endothelial cell growth medium), and growth factor kit (CC-3162) were purchased from Lonza. Dulbecco's phosphate-buffered salt solution 1× (DPBS, 1×) (21-031-CVR), 96-well microplate (9018), and Matrigel phenol-red free (356237) were purchased from Corning. TrypLE express enzyme (12605010) was purchased from Gibco™. HA10k-MSCA was synthesized by Holy Stone Healthcare.

HA10k (2000.0 mg, 4.975 mmol) was dissolved in 200 mL DDW and stirred for 2 hours. 320 mL DMSO was added into HA solution then wait for the solution to cool down to room temperature. The ethyl cyanohydroxyiminoacetate (Oxyma) (777.1 mg, 5.473 mmol) was dissolved in 20 mL DMSO and added to HA solution. After stirring for 15 mins, 0.68 equivalent (941.3 mg, 3.383 mmol) MSCA was dissolved in 20 mL of DMSO respectively and added to HA solution. After stirring 15 mins, 1555.5 μL (9.950 mmol) of N,N′-diisopropylcarbodiimide (DIC) was dissolved in 40 mL DMSO and added to HA solution. The final reaction mixture was stirred for 24 hours at room temperature. The solution of the product was dialyzed for 6 days. The first day was DMSO/DI water (1:1.5), the second day was DMSO/DI water (1:4), and the last four days were DI water twice a day. These results indicated the successful conjugation of HA-10k-MSCA.

The phenol red-free Matrigel (with a protein concentration of 9.2 mg/mL) was thawed by placing it overnight on ice and keeping the ice bucket in the refrigerator at 4 °C. The culture plates were pre-cooled by placing in the refrigerator for over 30 minutes and also kept on ice during the coating process. The required number of wells was coated with Matrigel in 100 μL per well was used for coating 96-well culture plates. The plate was left undisturbed in the biosafety cabinet for 10 minutes at room temperature. After 10 minutes, the Matrigel-coated plate was gently shifted to CO₂ incubator at 37 °C. It was left in the incubator for 30 minutes to allow for gelation of the Matrigel. After coating, 3×10\hat{ }4 HUVEC cells (p4) were seeded on to the Matrigel-coated wells of 96-well (60 μL per well) plate, followed by the addition of the medium containing 25, 50, 100 μM of HA4-MSCA, HA10k-MSCA or 15 μM Suramin (positive control) and then incubated at 37 °C for 16 hours. Subsequently, tube formation was observed under a microscope (Thermo Fisher) and the total tube length was measured by using Image J software (FIJI 1.46r, revised edition)

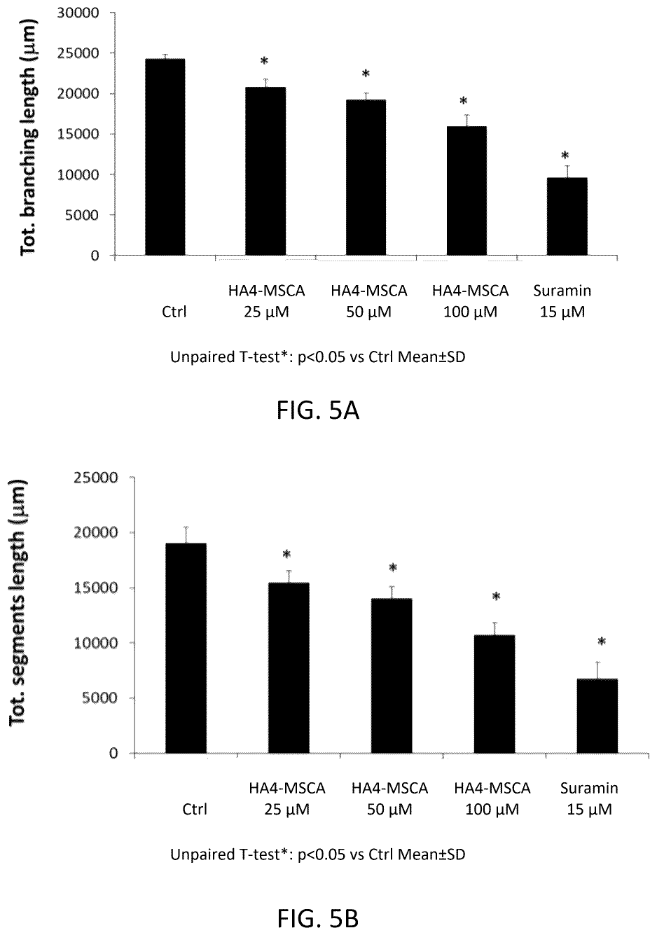

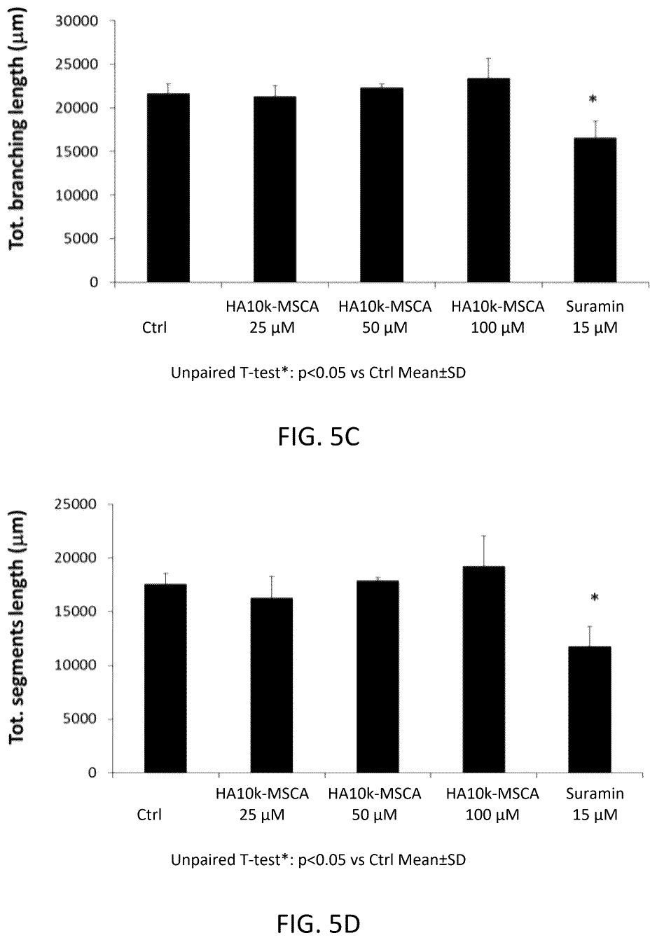

The results, as summarized in FIG. 4 and FIGS. 5A to 5D, indicate that that the treatment with the HA4-MSCA compound according to embodiments of the present disclosure results in fewer and/or smaller tubes formation, compared to the HA10k-MSCA compound. These results suggest that the present HA4-MSCA compound (at the specified concentration) inhibits tube formation, indicating a potential anti-angiogenic effect.

In particular, the total branch length provides a measurement of the extent of network formation. Results in FIG. 5A indicate that the total branch length in the HA4-MSCA treated groups is significantly reduced compared to a control group. This suggests that the present HA4-MSCA compound is capable of reducing the ability of the endothelial cells to form new vascular structures.

Further, the total segment length reflects the size of the tube structures. Results in FIG. 5B indicate that the total segment length in the HA4-MSCA treated groups is significantly reduced compared to a control group. This suggests that the present HA4-MSCA compound is capable of reducing the ability of HUVECs to form long or stable tube structures.

In contrast, the results in FIG. 5C to 5D indicate that the HA10k-MSCA compound is not capable of reducing the total segment length and total branch length in HUVECs.

The treatment with the present HA4-MSCA compound also suggests a dose-dependent inhibition. This indicates that higher concentrations of the present HA4-MSCA compound more effectively inhibit angiogenesis, potentially positioning the present HA4-MSCA compound as a candidate for anti-angiogenic therapies, such as in the treatment of conditions where excessive angiogenesis is a factor (e.g., wet AMD).

### Working Example 3

### Evaluation of Therapeutic Effect of HA2-MSCA and HA1-MSCA on Human Umbilical Vascular Endothelial Cell Tube Formation

This example was conducted using a protocol similar to working example 2 above. HA2-MSCA (AB8141) and HA1-MSCA (AB8198) were synthesized by Holy Stone Healthcare.

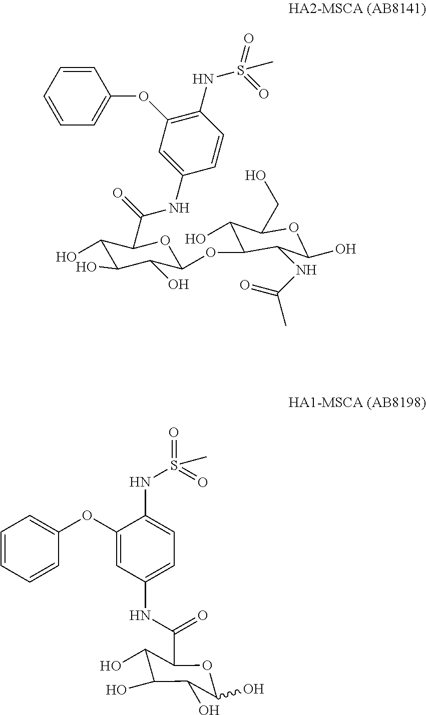 [SMILES: CC(=O)NC1C(O)OC(CO)C(O)C1OC1OC(C(=O)Nc2ccc(NS(C)(=O)=O)c(Oc3ccccc3)c2)C(O)C(O)C1O ; CS(=O)(=O)Nc1ccc(NC(=O)C2OC(O)C(O)C(O)C2O)cc1Oc1ccccc1]

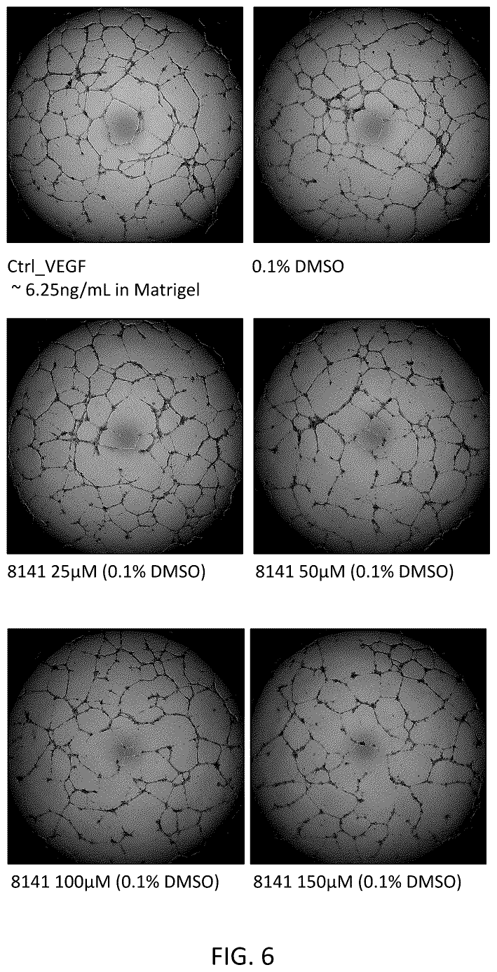

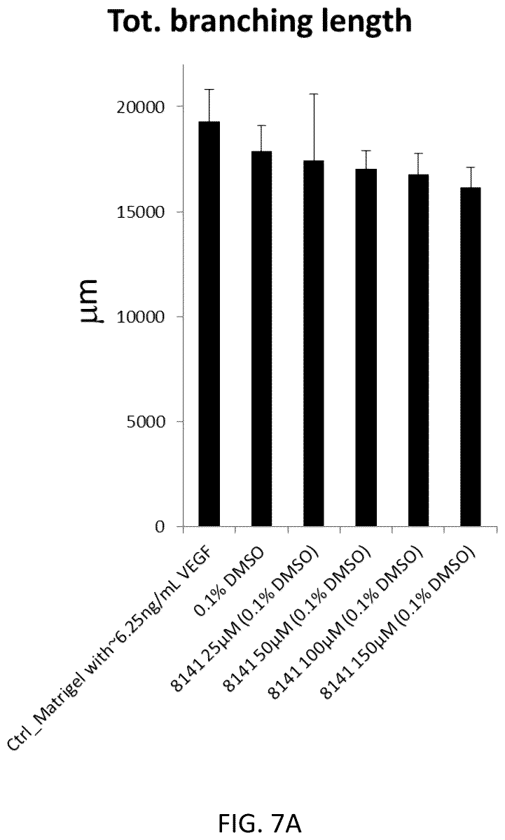

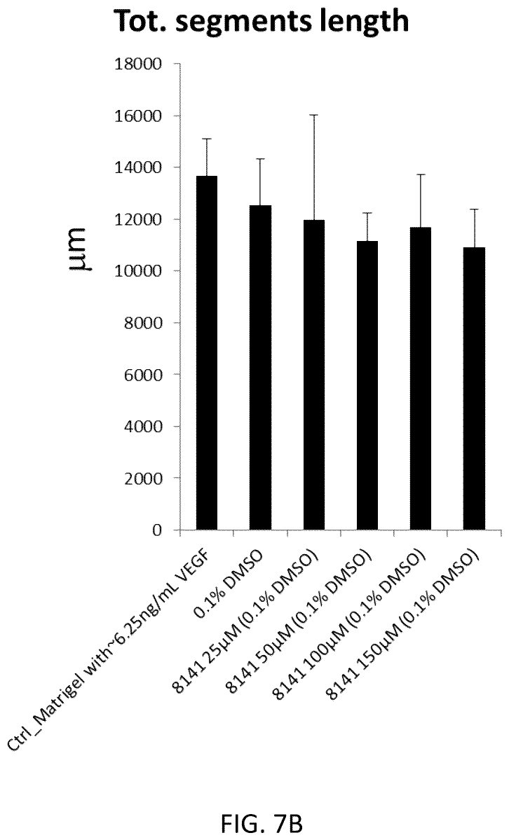

The results, as summarized in FIGS. 6, 7A, and 7B, indicate that that the treatment with the HA2-MSCA compound according to embodiments of the present disclosure results in fewer and/or smaller tubes formation, compared to the negative control (DMSO), in a dose-dependent manner. These results suggest that the present HA2-MSCA compound (at the specified concentration) inhibits tube formation, indicating a potential anti-angiogenic effect. This indicates that higher concentrations of the present HA2-MSCA compound more effectively inhibit angiogenesis, potentially positioning the present HA2-MSCA compound as a candidate for anti-angiogenic therapies, such as in the treatment of conditions where excessive angiogenesis is a factor (e.g., wet AMD).

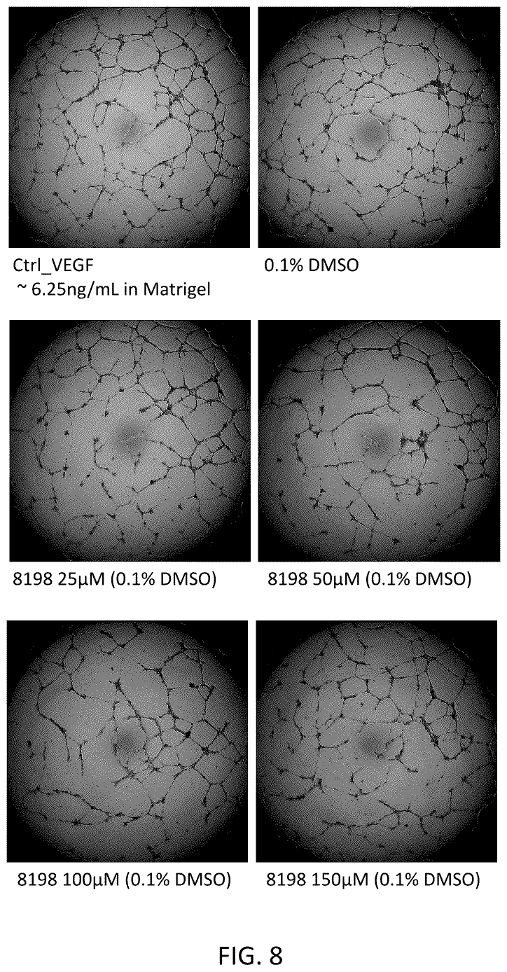

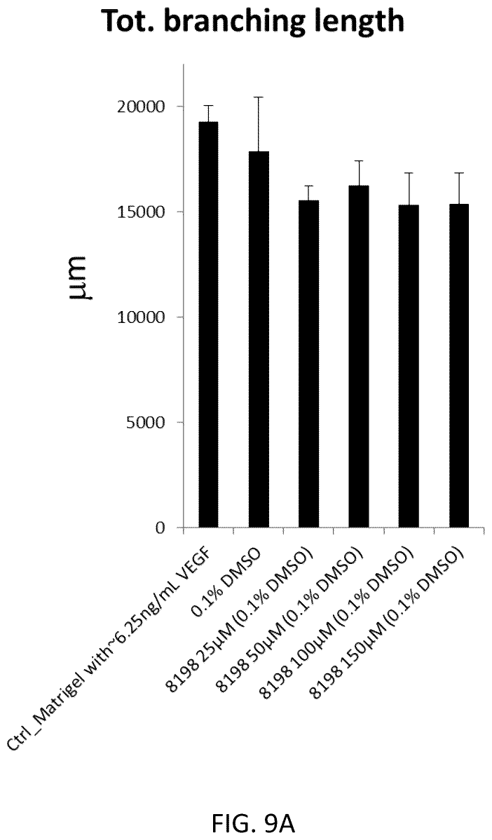

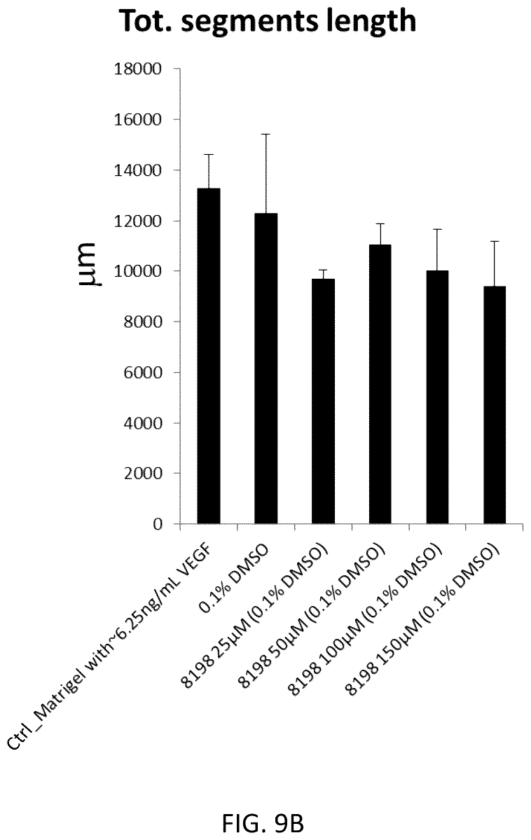

Similarly, the results summarized in FIGS. 8, 9A, and 9B, indicate that that the treatment with the HA1-MSCA compound according to embodiments of the present disclosure results in fewer and/or smaller tubes formation, compared to the negative control (DMSO), in a dose-dependent manner. These results suggest that the present HA1-MSCA compound (at the specified concentration) inhibits tube formation, indicating a potential anti-angiogenic effect. This indicates that higher concentrations of the present HA1-MSCA compound more effectively inhibit angiogenesis, potentially positioning the present HA1-MSCA compound as a candidate for anti-angiogenic therapies, such as in the treatment of conditions where excessive angiogenesis is a factor (e.g., wet AMD).

It will be understood that the above description of embodiments is given by way of example only and that various modifications may be made by those with ordinary skill in the art. The above specification, examples, and data provide a complete description of the structural and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. An organic compound according to formula 1: 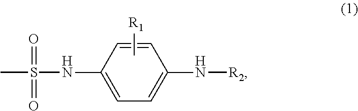
where R₁ represents a phenyl group, a phenoxy group, a (C1-C30) alkyl group, a (C2-C30) alkenyl group, a (C2-C30) alkynyl group or a (C1-C30) alkoxy group; and R₂ represents a hydroxyl group, carboxyl group or a saccharide.

2. The organic compound of claim 1, wherein the saccharide comprises one to four saccharide units.

3. The organic compound of claim 2, wherein the saccharide units are at least one selected from the group consisting of glucose, fructose, galactose, glucuronic acid, sucrose, lactose, maltose, glycosaminoglycan, isomaltotriose, panose, nigerotriose, maltotriose, melezitose, maltotriulose, raffinose, kestose, ychnose, maltotetraose, nigerotetraose, nystose, sesamose and stachyose, and derivatives thereof.

4. The organic compound of claim 1, wherein the organic compound is administered to a patient via an intravitreal injection (IVT).

5. A method for preventing and/or treating an ocular disorder, comprising administering to a patient in need thereof an effective amount of an organic compound of claim 1.

6. The method of claim 5, wherein the effective concentration of the organic compound is 1 to 100 mg/mL of the organic compound.

7. The method of claim 5, wherein the organic compound is administered to the patient via intravitreal injection (IVT).

8. The method of claim 5, wherein the ocular disorder is dry eye syndrome, dry AMD, wet AMD, macular edema, macular edema following retinal vein occlusion, retinal vein occlusion (RVO), central retinal vein occlusion (CRVO), branch retinal vein occlusion (BRVO), diabetic macular edema (DME), choroidal neovascularization (CNV), iris neovascularization, neovascular glaucoma, proliferative vitreoretinopathy (PVR), optic disc neovascularization, corneal neovascularization, or proliferative diabetic retinopathy.

9. The method of claim 8, wherein the ocular disorder is wet AMD.
